(19) United States
(12) Patent Application Publication
(10) Pub. No.: US 2026/0248415 A1
(43) Pub. Date: Aug. 27, 2026

(54) GAIT ANALYSIS METHOD AND, SYSTEM, AND APPARATUS DEVICE FOR IMPROVING WALKING OBSTACLE

(71) Applicant: BEIJING DALAI TECHNOLOGY CO., LTD, Beijing (CN)

(72) Inventors: Kailiang WANG, Beijing (CN); Biao CHEN, Beijing (CN); Jun FENG, Beijing (CN); Wei HU, Beijing (CN); Yuan MENG, Beijing (CN)

(21) Appl. No.: 18/870,429

(22) PCT Filed: Aug. 31, 2023
(86) PCT No.: PCT/CN2023/116047

§ 371 Date: Nov. 28, 2024

(30) Foreign Application Priority Data
Sep. 22, 2022 (CN) 202211157651.2

(51) Int. Cl. A61B 5/11; A61B 5/00; A61B 5/107

(52) U.S. Cl. CPC A61B 5/112; A61B 5/1071; A61B 5/6807; A61B 5/7257; A61B 2562/0219; A61B 2562/0247

(57) ABSTRACT

The present disclosure provides a gait analysis method and system, and an apparatus for improving a walking obstacle. The method includes: acquiring three-axis acceleration data and three-axis angular velocity data collected by an inertial sensor, and acquiring pressure data collected by a pressure sensor; performing a quaternion operation on the three-axis acceleration data and the three-axis angular velocity data to acquire three-axis acceleration and a three-axis Euler angle corresponding to a ground coordinate system; identifying a swinging state and a stationary state of feet based on the three-axis acceleration data and the pressure data; analyzing based on the three-axis acceleration, the three-axis Euler angle, and the swinging state and the stationary state of the feet to acquire gait parameters related to a straight walking process, a turning processing, a feet to ground angle, and a feet tremor; and uploading the gait parameters to a server.

The present disclosure claims priority to Chinese Patent Application No. 2022111576512, filed on Sep. 22, 2022 and entitled “GAIT ANALYSIS METHOD AND SYSTEM, AND APPARATUS FOR IMPROVING WALKING OBSTACLE” to the China National Intellectual Property Administration, the disclosure of which is herein incorporated by reference in its entirety.

### TECHNICAL FIELD

The present disclosure relates to the field of gait analysis technologies and, in particular, to a gait analysis method and system, and an apparatus for improving a walking obstacle.

### BACKGROUND

In today's health care system, a doctor generally needs to watch a patient's gait to determine severity of symptoms in a Parkinson's patient. A specific method used is to monitor and observe the patients' gaits in real-time, such as a step length, a step velocity, and a turning velocity. In this way, the severity of the patient's symptoms can be determined, and different treatment plans can be developed for the patient or the patient's recovery level can be evaluated, thereby providing an objective basis for developing a rehabilitation treatment plan and evaluating rehabilitation efficacy. The current method requires the doctor to observe with naked eyes, and conclusions acquired by different doctors may vary slightly, lacking objectivity. The doctor needs to constantly monitor and observe the patient's gait, which requires a lot of time, increasing workloads of the doctor. For the patient, a process of analyzing a gait needs to be carried out in a hospital, and it requires a lot of time and efforts on a way to seek medical treatment, increasing a burden on the patient.

Therefore, how to provide the doctor with objective and comprehensive motion data of the patient's feet, and reduce the burden on the doctor and the patient, has become an urgent technical problem and a constantly researched focus for those skilled in the art.

### SUMMARY

The present disclosure aims to provide a gait analysis method that may provide objective and comprehensive motion data of a patient's feet to provide an objective basis for formulating a rehabilitation treatment plan and evaluating rehabilitation efficacy. Collection of the motion data is not limited by a venue, which may reduce a burden on a doctor and a patient.

To solve the above problem in the prior art, the present disclosure provides the following technical solutions.

In a first aspect, the present disclosure provides a gait analysis method, including the following steps:
acquiring three-axis acceleration data and three-axis angular velocity data collected by an inertial sensor;
performing a quaternion operation on the three-axis acceleration data and the three-axis angular velocity data to acquire three-axis acceleration and a three-axis Euler angle corresponding to a ground coordinate system;
identifying a swinging state and a stationary state of feet;
analyzing based on the three-axis acceleration, the three-axis Euler angle, and the swinging state and the stationary state of the feet to acquire gait parameters related to the straight walking process, a turning processing, a feet to ground angle, and a feet tremor; and
uploading types of the gait parameters and numerical values corresponding to the gait parameters to a server.

In a second aspect, the present disclosure provides a detection system for a frozen gait, including:
a feet data acquiring module configured to acquire three-axis acceleration data and three-axis angular velocity data collected by an inertial sensor;
a feet data calculating module configured to perform a quaternion operation on the three-axis acceleration data and the three-axis angular velocity data to acquire three-axis acceleration and a three-axis Euler angle corresponding to a ground coordinate system;
a feet state identifying module configured to identify a swinging state and a stationary state of feet;
a gait parameter generating module configured to analyze based on the three-axis acceleration, the three-axis Euler angle, and the swinging state and the stationary state of the feet to acquire gait parameters related to the straight walking process, a turning processing, a feet to ground angle, and a feet tremor; and
a gait parameter uploading module configured to upload types of the gait parameters and numerical values corresponding to the gait parameters to a server.

In a third aspect, the present disclosure provides an apparatus for improving a walking obstacle, including shoes, a terminal device, and a server, where
the shoes have an inertial sensor, a pressure sensor, a vibration element, an electrical stimulation element, and a communication module built in, where
the inertial sensor is configured to collect three-axis acceleration data and three-axis angular velocity data;
the pressure sensor is configured to collect pressure data;
the communication module is configured to establish a communication link with the terminal device, and transmit the three-axis acceleration data, the three-axis angular velocity data, and the pressure data to the terminal device;
the server is provided with a processor, a memory, and a communication unit, where the memory is configured to store a program, and the three-axis acceleration data, the three-axis angular velocity data, and the pressure data from the terminal device, the processor calls the program stored in the memory to perform the gait analysis method according to any in the first aspect, and the communication unit is configured to establish the communication link with the terminal device; and
the terminal device controls the vibration element and the electrical stimulation element, where when a patient experiences an abnormal gait, the terminal device controls the vibration element to output vibration stimulation and/or the electrical stimulation element to output electrical stimulation.

In a fourth aspect, the present disclosure provides a computer readable storage medium, including a program, where the program, when performed by a processor, is configured to perform the gait analysis method according to any in the first aspect.

Compared with the prior art, the present disclosure has the following advantages. The gait parameters in the present disclosure have a relatively wide coverage range, which may more comprehensively and accurately determine symptoms of a Parkinson's patient, thereby providing an objective basis for formulating the rehabilitation treatment plan and evaluating the rehabilitation efficacy. In the present disclosure, a patient merely needs to wear gait-monitoring shoes, so that the gait parameters that can be determined by the doctor can be automatically acquired without involvement of a doctor. In addition, there are no restrictions on a venue, so gait data can be collected when the patient is at home. After the collected gait data is uploaded to the server, the returned gait parameters are sent to the doctor, and then the doctor can determine the patient's symptoms, thereby reducing workloads of the doctor. At the same time, it can save the patient with mobility difficulties a need to travel to and from a hospital, thereby reducing a burden on the patient.

Further effects of the above non-conventional implementation manner will be explained in combination with specific implementation manners in the following.

### BRIEF DESCRIPTION OF THE DRAWINGS

For clearer description of embodiments in the present disclosure or technical solutions in the prior art, drawings to be referred to for the description of the embodiments or the prior art are briefly introduced below. Apparently, the drawings in the description below merely illustrate some embodiments recorded in the present disclosure, and those skilled in the art may also derive other drawings according to these drawings without creative labors.

FIG. 1 is a schematic flowchart of a method embodiment according to the present disclosure;

FIG. 2 is a schematic diagram of waveform change after preprocessing three-axis acceleration and waveform generation of a first state variable in a method embodiment according to the present disclosure;

FIG. 3 is a schematic diagram of waveform generation of a second state variable in a method embodiment according to the present disclosure;

FIG. 4 is a schematic diagram of variations of an Euler angle waveform during a correction process in a method embodiment according to the present disclosure;

FIG. 5 is a schematic diagram of a change in maximum points and minimum points selected when extracting a gait during a turning process in a method embodiment according to the present disclosure;

FIG. 6 is a schematic diagram of segmented extraction of Euler angle data in a Z-axis direction in a method embodiment according to the present disclosure;

FIG. 7 is a schematic structural diagram of a system implementation according to the present disclosure; and

FIG. 8 is a schematic structural diagram of an apparatus for improving a walking obstacle according to the present disclosure.

### DETAILED DESCRIPTION

Exemplary embodiments will be described in detail herein, with examples shown in drawings. When the following description refers to the drawings, same numbers in different drawings represent the same or similar elements unless otherwise indicated. Implementation manners described in the following exemplary embodiments do not represent all implementation manners consistent with the present disclosure. Rather, they are merely examples of apparatus and methods consistent with certain aspects of the present disclosure, as detailed in the appended claims.

Terms used in the present disclosure are merely for a purpose of describing specific embodiments and are not intended to limit the present disclosure. Singular forms of “one”, “the”, and “this” used in the present disclosure and the appended claims are also intended to include a majority form, unless context clearly indicates other meanings. It should also be understood that a term “and/or” used in the present disclosure refers to and includes any or all possible combinations of one or more associated listed items.

It should be understood that although terms first, second, third, and the like, may be used in the present disclosure to describe various information, the information should not be limited to these terms. These terms are merely used to distinguish information of the same type from each other. For example, without departing from a scope of the present disclosure, first information may also be referred to as second information, and similarly, the second information may also be referred to as the first information.

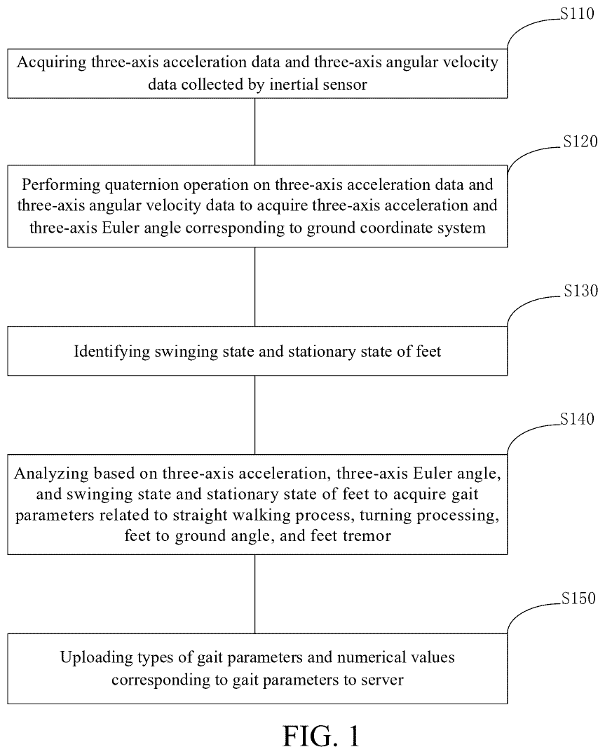

Based on shortcomings of the prior art, embodiments of the present disclosure provide a specific implementation manner of a gait analysis method. Referring to FIG. 1, the method specifically includes the following steps.

In S110, three-axis acceleration data and three-axis angular velocity data collected by an inertial sensor are acquired.

In S120, a quaternion operation is performed on the three-axis acceleration data and the three-axis angular velocity data to acquire three-axis acceleration and a three-axis Euler angle corresponding to a ground coordinate system.

In S130, a swinging state and a stationary state of feet are identified.

In S140, an analysis is performed based on the three-axis acceleration, the three-axis Euler angle, and the swinging state and the stationary state of the feet to acquire gait parameters related to the straight walking process, a turning processing, a feet to ground angle, and a feet tremor.

More precisely, in a step S140, an analysis is performed based on the swinging state and the stationary state to acquire the gait parameters related to the straight walking process;
an analysis is performed based on the three-axis Euler angle to acquire the gait parameters related to the turning process;
an analysis is performed based on the three-axis Euler angle and the swinging state to acquire the gait parameters related to the feet to ground angle; and
an analysis is performed based on the three-axis acceleration and the three-axis Euler angle to acquire the gait parameters related to the feet tremor.

In S150, types of the gait parameters and numerical values corresponding to the gait parameters are uploaded to a server.

Specifically, an inertial sensor includes an accelerometer and a gyroscope, with a sampling frequency of fs=50 Hz. The accelerometer is used to collect the three-axis acceleration data, and the gyroscope is used to collect the three-axis angular velocity data. For ease of collection, the accelerometer and the gyroscope may be installed in shoes to make gait-detecting shoes. During a gait-acquiring process, a patient merely needs to wear the gait-detecting shoes to complete the collection of the three-axis acceleration data and the three-axis angular velocity data. The three-axis acceleration data includes acceleration signals in an X-axis, a Y-axis, and a Z-axis, while the three-axis angular velocity data includes angular velocity signals in the X-axis, the Y-axis, and the Z-axis. The quaternion operation can complete rotation of 3D coordinates to rotate the 3D coordinates that need to be rotated to a desired position. After the patient walks for a certain period of time, all gait parameters can be calculated under the premise of determining the patient's feet swing and stillness. Moreover, the gait parameters may be divided into four categories: a first category is the gait parameters related to the straight walking process, a second category is the gait parameters related to the turning process, a third category is the gait parameters related to the feet to ground angle, and a fourth category is the gait parameters related to the patient's feet tremor. Further, all gait parameters related to the patient's feet are comprehensively reflected. Finally, the calculated gait parameters of the above four categories may be compiled into a table form and uploaded to the server as a gait analysis report. The doctor can view it through a mobile terminal that communicates with the server.

In this embodiment, a coverage range of the gait parameters is relatively wide, which may more comprehensively and accurately determine symptoms of a Parkinson's patient, thereby providing an objective basis for formulating a rehabilitation treatment plan and evaluating rehabilitation efficacy. A patient merely needs to wear gait-monitoring shoes, so that the gait parameters that can be determined by the doctor can be automatically acquired without involvement of a doctor. In addition, there are no restrictions on a venue, so gait data can be collected when the patient is at home. After the collected gait data is uploaded to the server, the returned gait parameters are sent to the doctor, and then the doctor can determine the patient's symptoms, thereby reducing workloads of the doctor. At the same time, it can save the patient with mobility difficulties a need to travel to and from a hospital, thereby reducing a burden on the patient.

In one embodiment of the present disclosure, identifying the swinging state and the stationary state of the feet includes:
setting a critical action threshold and representing it using a state variable stationary, where when the state variable stationary is 1, it indicates that the feet are in the stationary state, and when the state variable stationary is 0, it indicates that the feet are in the swinging state.

Identification of the swinging state and the stationary state of the feet may be performed in the same two methods as follows.

The first method is to preprocess the three-axis acceleration to acquire an acceleration signal, where if the acceleration signal exceeds the critical action threshold, it is considered to be in the swinging state, and if the acceleration signal is below the critical action threshold, it is considered to be in the stationary state, and a first state variable stationary1 is acquired.

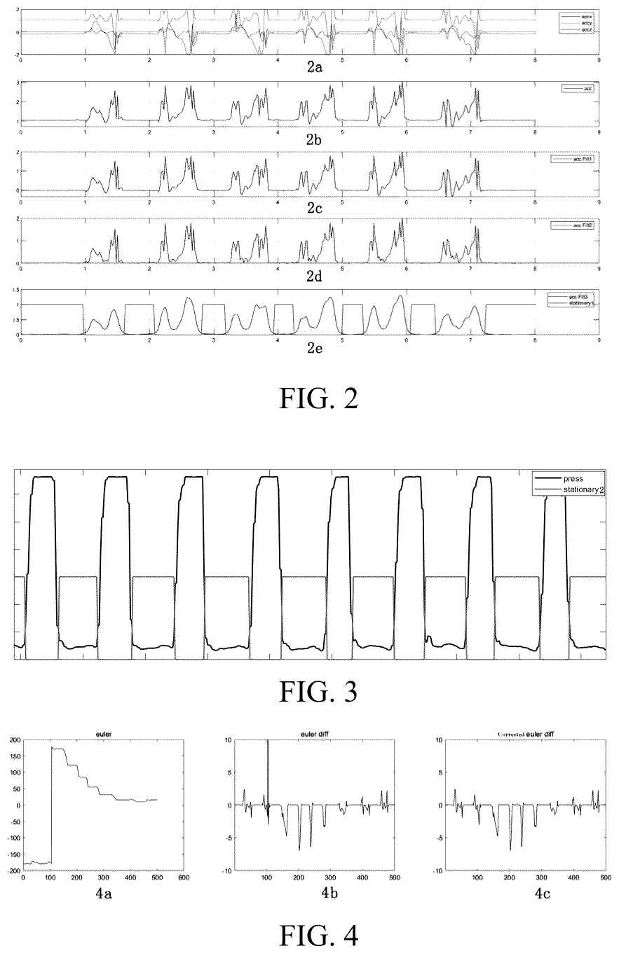

In this method, the critical action threshold may be set to 0.05, where 0.05 is a numerical value of the acceleration signal, which is a numerical value of a vertical axis of a waveform shown in 2e of FIG. 2. The first state variable thereof stationary1 is represented by the formula:

$$stationary 1 = \{ \begin{matrix}1 & ( {acc}_{Filt 3} \leq 0.05 ) \\ 0 & ( {acc}_{Filt 3} > 0.05 )\end{matrix} ;$$

where stationary1 is the first state variable, 1 is the stationary state, and 0 is the swinging state.

The second method is to acquire pressure data press collected by a pressure sensor, where if the pressure data press exceeds the critical action threshold, it is considered to be in the swinging state, and if the pressure data press below the critical action threshold, it is considered to be in the stationary state, and a second state variable stationary2 is acquired.

It should be noted that the pressure data press reflects a size of a voltage value. When the shoes are stepped down, pressure increases and resistance in a pressure sensor decreases. In the case of constant current, a pressure value will decrease. Therefore, an actual pressure value is inversely proportional to a numerical value of the pressure sensor.

In this method, the critical action threshold can be set to 600, where 600 is a numerical value of the pressure data collected by the pressure sensor. The pressure data collected by the pressure sensor is a waveform (wave shape) shown in FIG. 3. A waveform of the second state variable stationary2 is a regular sawtooth shape, and the second state variable thereof stationary2 is represented by a formula:

$$stationary 2 = \{ \begin{matrix}1 & ( press < 600 ) \\ 0 & ( press \geq 600 )\end{matrix} ;$$

where stationary2 is the second state variable, 1 is the stationary state, and 0 is the swinging state.

An OR operation is performed on the first state variable stationary1 and the second state variable stationary2, and the swinging state and the stationary state of the feet are identified based on an operation result. A process thereof is represented by a formula:

$$stationary = stationary 1 \vert \vert stationary 2.$$

In this embodiment, two state variables are used to identify the swinging state and the stationary state of the feet. Only when the first state variable stationary1 and the second state variable stationary2 are both 0, it is identified as the swinging state, while in other cases, it is identified as the stationary state. In this way, it can effectively avoid misidentification of the swinging state, and improve accuracy of identifying the swinging state of the feet, thereby providing a more accurate triggering basis for a subsequent process.

In one embodiment of the present disclosure, specific steps of preprocessing the three-axis acceleration to acquire the acceleration signal include the following.

A waveform of the three-axis acceleration shown in 2a of FIG. 2 is referred to, which includes acceleration waveforms in three directions of the X-axis, the Y-axis, and the Z-axis.

A vector sum of the three-axis accelerations is calculated to acquire an original acceleration signal, whose waveform is shown in 2b of FIG. 2.

A calculation formula thereof is as follows:

$$acc = \sqrt{{acc}_{x}^{2} + {acc}_{y}^{2} + {acc}_{z}^{2}} ;$$

where acc is an original acceleration signal, accₓ is acceleration in the X-axis, acc_{y} is acceleration in the Y-axis, and acc_{z} is acceleration in the Z-axis.

A high-pass filtering process is performed on the original acceleration signal with a cutoff frequency of 0.01 Hz to acquire an acceleration signal filtered once. After the high-pass filtering process is completed, a waveform thereof is shown in 2c in FIG. 2.

A process thereof is represented by a formula:

$${acc}_{Filt 1} = {Filt}_{0.01}^{high} ( acc ) ,$$

where acc_{Filt1} is an acceleration signal filtered once.

A low-pass filtering process with a cutoff frequency of 5 Hz is performed to acquire a final acceleration signal after performing an absolute value calculation on the acceleration signal filtered once. A waveform after the absolute value operation is shown in 2d of FIG. 2, and a waveform after the low-pass filtering process is shown in 2e of FIG. 2.

A process thereof is represented by a formula:

$${acc}_{Filt 1} = \vert {acc}_{Filt 1} \vert ,$$

where acc_{Filt2} is an acceleration signal after the absolute value operation.

$${acc}_{Filt 3} = {Filt}_{5}^{low} ( {acc}_{Filt 2} ) ,$$

where acc_{Filt3} is an acceleration signal filtered twice.

In one embodiment of the present disclosure, the gait parameters related to the straight walking process include the number of steps step_n, a step lengthstep_length, a step frequency step_freq, a step velocitystep_vel, and a gait variation coefficient step_SD.

The number of steps step_n is acquired through the following method.

A first-order differential operation is performed on the state variable stationary to acquire a differential vector stationary_{diff}. The differential vector stationary diff with a numerical value of −1 is identified as a starting moment for each step, the differential vector stationary diff with a numerical value of 1 is identified as an ending moment for each step, and the number of differential vectors stationary_{diff} with numerical values of −1 and 1 are respectively counted. A minimum value from a counting result is selected to acquire the number of steps step_n in the gait parameters.

The step lengthstep_length is acquired through the following method.

A starting point idxₛₜₐᵣₜ where all numerical values of the differential vector stationary_{diff} are −1 and an ending point idx_{end} where all numerical values of the differential vector stationary_{diff} are 1 are selected.

A velocity velfrom the starting point idxₛₜₐᵣₜ to the ending point idx_{end} is calculated.

A starting position pos_{i,start} for each step is calculated based on the velocity vel at each moment before the starting point idxₛₜₐᵣₜ, and an ending position pos_{i,end} for each step is calculated based on the velocity vel at each moment before the ending point idx_{end}.

The velocity vel is calculated through the following formula:

$$v e {l}_{t} = a c {c}_{t} \times Δ t + v e {l}_{t - 1} ;$$

where velₜ is a velocity at a certain moment, acct is acceleration at a certain moment, Δ t is interval time, and Δt=1/fs=0.02, and velₜ₋₁ is a velocity at a previous moment.

To reduce errors, an offset vel_{drift} in the velocity vel is removed before calculating a position at a certain moment.

The offset vel_{drift} is calculated through the following formula:

$$v e {l}_{end} = \frac{v e {l}_{end}}{N - 1} \times enum ;$$

where N is a data length, enum is a vector with a value range of (0, 1, 2, 3, . . . , N−1).

The starting position pos_{i,start} and the ending position pos_{i,end} are both calculated using the following formula:

$${pos}_{t} = {vel}_{t} \times Δ t + {pos}_{t - 1} ;$$

where posₜ is a displacement at a certain moment, velₜ is a velocity at a certain moment, Δt is interval time, and Δt=1/fs=0.02, and posₜ₋₁ is a displacement at a certain moment.

A difference value between the ending position pos_{i,end} and the starting position pos_{i,start} is calculated to acquire the step size step_length in the gait parameters.

The step size step_length is represented througha formula:

$$step\_length = \frac{1}{step\_n} \times {\sum }_{i = 1}^{step \_ n} ( p o {s}_{i , end} - po {s}_{i , start} ) .$$

The step frequency step_freq is acquired through the following method.

A data length from a starting point idx_{i,start} for each step to a starting point idx_{i+1,start} for a next step during a swinging process is calculated to acquire a data length Nᵢ for each step.

A process thereof is represented through a formula:

$${N}_{i} = {idx}_{i + 1 , start} - {idx}_{i , start} .$$

A ratio of the data length Nᵢ to a sampling frequency fs for each step is calculated and an average value is taken to acquire a swinging period step_T.

The swinging period step_T is calculated through the following formula:

$$step\_T = \frac{1}{step\_n} \times {\sum }_{i = 1}^{step \_ n} \frac{{N}_{i}}{fs} .$$

A reciprocal of the swinging period step_T is calculated to acquire a step frequency step_freq in the gait parameters.

The step frequency step_freq is calculated through the following formula:

$$step\_freq = \frac{1}{step\_T} .$$

A step velocity step_vel is acquired through the following method.

A ratio of a total step size step_length to a total data length N at the sampling frequency fs within unit time is calculated to acquire a step velocity step_vel in the gait parameters.

It is represented through a formula:

$$step\_vel = \frac{fs}{N} \times {\sum }_{i = 1}^{N} {step\_length}_{i} ;$$

where fs is a sampling frequency, and N is a data length.

A gait variation coefficient step_SD is acquired through the following method.

A ratio of a standard deviation of all step lengthsstep_length to a step lengthstep_length is calculated to acquire a gait variation coefficient step_SD in the gait parameters.

It is represented through a formula:

$$step\_SD = \sqrt{\frac{{\sum }_{i = 1}^{step \_ n} {( {step\_length}_{i} - step\_length )}^{2}}{step\_n}} \times \frac{1}{step\_length} .$$

A normal gait and an abnormal gait may be distinguished by the gait variation coefficient step_SD.

In one embodiment of the present disclosure, the gait parameters related to the turning process include the number of turning steps turn_n, a turning angle angle_turn, and a turning velocity angle_turn_vel.

When calculating the gait parameters related to the turning process, it is necessary to first separate the turning process from an entire walking process, and then separate each step included in the turning process separately. A specific implementation manner thereof is as follows.

The three-axis angular velocity data is selected to perform the quaternion operation to acquire an Euler angle eulerₓ in an X-axis, and the Euler angle eulerₓ in the X-axis reflects a yaw angle during a feet motion process.

A first-order differential operation is performed on the Euler angle eulerₓ to acquire a differential Euler angle euler_{diff}.

A waveform of the Euler angle eulerₓ in the X-axis direction is shown in 4a of FIG. 4. It can be seen that the Euler angle eulerₓ undergoes a sudden change when it is greater than 180° or less than −180°. After performing the first-order differential operation on the Euler angle eulerₓ, a waveform shown in 4b of FIG. 4 is acquired. There is still a sudden change in this waveform, so it is necessary to correct the differential Euler angle euler_{diff} of the Euler angle eulerₓ. A correction process thereof is represented through the following formula:

$${euler}_{t , diff} = \{ \begin{matrix}{euler}_{t + 1} - {euler}_{t} - 360 ( {euler}_{t , diff} > 180 ) \\ {euler}_{t + 1} - {euler}_{t} + 360 ( {euler}_{t , diff} < - 180 )\end{matrix} ,$$

A waveform of a corrected differential Euler angle euler_{diff} is shown in 4c of FIG. 4. The waveform shown in 4c of FIG. 4 is acquired by linear fitting multiple discrete data points.

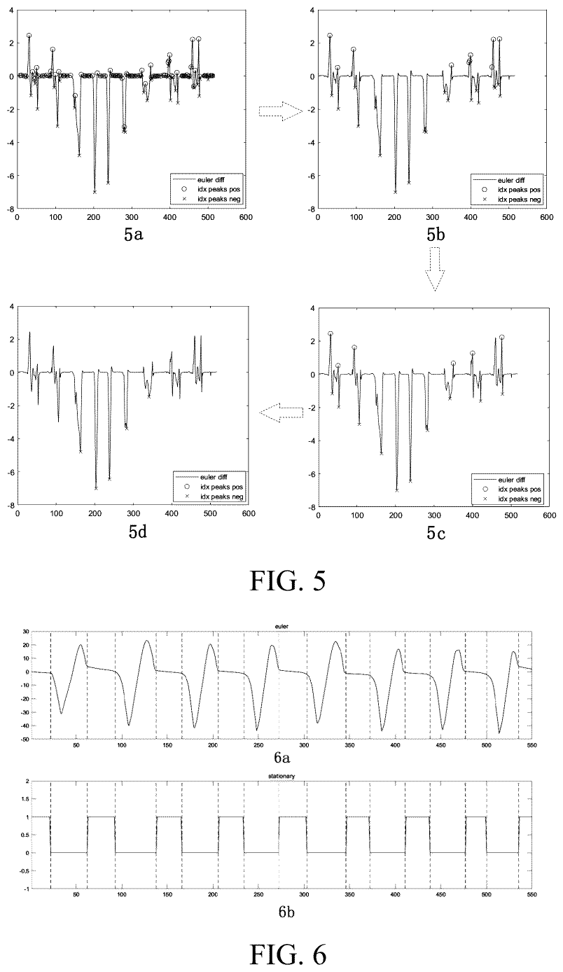

All maximum points idx_peaksₚₒₛ and minimum points idx_peaks_{neg} in the differential Euler angle euler_{diff} are extracted, and after they are selected, peak points circled in a waveform shown in 5a of FIG. 5 are acquired.

An analysis is performed based on all the maximum points idx_peaksₚₒₛ and the minimum points idx_peaks_{neg} in the differential Euler angle euler_{diff} to acquire the starting point idxₛₜₐᵣₜ and the ending point idx_{end} for each step. A specific acquiring method thereof is as follows.

Firstly, an extreme point with a peak value less than a first peak threshold in the maximum points idx_peaksₚₒₛ and an extreme point with a peak value greater than a second peak threshold in the minimum points idx_peaks_{neg} are deleted. Selection of the first peak threshold and the second peak threshold should refer to vertical axis numerical values in 4a of FIG. 4. For example, the first peak threshold may be selected as 0.5, and the second peak threshold may be selected as −0.5. After deletion, peak points circled in a waveform shown in 5b of FIG. 5 may be acquired.

Secondly, the maximum points idx_peaksₚₒₛ or the minimum points idx_peaks_{neg} with a period interval less than a set period threshold are divided into a group, and the maximum points idx_peaksₚₒₛ or the minimum points idx_peaks_{neg} with a highest absolute peak value in each group are retained. A set period threshold is selected based on horizontal axis numerical values of 5a in FIG. 5. For example, the set period threshold is set to 15 to delete multiple maximum points idx_peaksₚₒₛ or minimum points idx_peaks_{neg} appearing on a peak, acquire clearer swinging points, and separate the turning process from the entire walking process, resulting in peak points circled in a waveform shown in 5c of FIG. 5.

Thirdly, a data segment between any maximum point idx_peaksₚₒₛ and any minimum point idx_peaks_{neg} is selected as a gait determination segment, where if there are consecutive data points in the gait determination segment where a value of the differential Euler angle euler_{diff} is less than a fourth threshold and the number is less than N, then selected maximum points idx_peaksₚₒₛ and minimum points idx_peaks_{neg} are generated in a same step. The fourth threshold may be set to 0.1, N may be set to 5, and each step during the turning process is separated separately in the final.

Fourthly, an order of the maximum points idx_peaksₚₒₛ and the minimum points idx_peaks_{neg} in the same step is determined.

Finally, consecutive data points where the value of the differential Euler angle euler_{diff} is less than the fourth threshold and the number exceeds M are searched left based on the maximum points idx_peaksₚₒₛ or the minimum points idx_peaks_{neg} sorted first, and a data point discovered first is selected as the starting point idxₛₜₐᵣₜ for this step.

Consecutive data points where the value of the differential Euler angle euler_{diff} is less than the fourth threshold and the number exceeds M are searched right based on the maximum points idx_peaksₚₒₛ or the minimum points idx_peaks_{neg} sorted last, and a data point discovered first is selected as the ending point idx_{end} for this step.

It should be noted that the preferred number of M is 5, which means that in a process of selecting the starting point idxₛₜₐᵣₜ and the ending point idx_{end}, it is necessary to find 5 consecutive data points where the value of the differential Euler angle euler_{diff} is less than 0.1. These 5 data points indicate that a gait changes from stationary to moving or from moving to stationary during a walking process, ensuring that a data point discovered first is the starting point idxₛₜₐᵣₜ or the ending point idx_{end} of this step in practice, thereby ensuring accuracy of identifying the starting point idxₛₜₐᵣₜ and the ending point idx_{end} for each step during the turning process to avoid identification errors.

The number of turning steps turn_n is acquired through the following method.

A difference value between an Euler angle eulerₓ (idx_{end}) corresponding to the end point idx_{end} and an Euler angle eulerₓ (idxₛₜₐᵣₜ) corresponding to the starting point idxₛₜₐᵣₜ is calculated to acquire an angle difference A. The maximum points idx_peaksₚₒₛ and the minimum points idx_peaks_{neg} are removed if an absolute value of the angle difference A is lower than a first threshold. The minimum points idx_peaks_{neg} are removed if an absolute value of the angle difference A is higher than a second threshold. The starting point idxₛₜₐᵣₜ and the ending point idx_{end} for each step acquired are traversed with a rule of removing the maximum points idx_peaksₚₒₛ if an absolute value of the angle difference A is lower than a third threshold. The first threshold may be set to 15, the second threshold to 15, and the third threshold to −15 to acquire peak points circled in a waveform shown in 5d of FIG. 5. The total number of remaining maximum points idx_peaksₚₒₛ and minimum points idx_peaks_{neg} is counted to acquire the number of turning steps turn_n in the gait parameters.

The turning angle angle_turn is acquired through the following method.

A sum of the difference value between the Euler angle euler (idx_{end}) corresponding to the ending point idx_{end} and the Euler angle euler (idxₛₜₐᵣₜ) corresponding to the starting point idxₛₜₐᵣₜ for each of the number of turning steps turn_n is calculated and an average value is taken to acquire a turning angle angle_turn in the gait parameters.

It is represented through a formula:

$$angle\_turn = \frac{1}{turn\_n} \times {\sum }_{i = 1}^{turn \_ n} [ euler ( {idx}_{i , end} ) - euler ( {idx}_{i , start} ) ] .$$

A turning speed angle_turn_vel is acquired through the following method.

A ratio of the number of data points Nᵢ in a turning process for each of the number of turning steps turn_n to the sampling frequency fs is calculated to acquire turning time for each step, and a ratio of the turning angle angle_turn to the turning time for each step is calculated and an average value is taken to acquire a turning velocity angle_turn_vel in the gait parameters.

It is represented through a formula:

$$angle\_turn \_vel = \frac{1}{turn\_n} \times {\sum }_{i = 1}^{turn \_ n} ( euler ( {idx}_{i , end} ) - euler ( {idx}_{i , start} ) ) \times \frac{fs}{{N}_{i}} ) ;$$

where, Nᵢ=idx_{i,end}−idx_{i,start}.

In one embodiment of the present disclosure, the gait parameters related to the feet to ground angle include a heel landing angle angle_heel_strike, a toe off ground angle angle_toe_off, a swinging phase swing_phase, and a standing phase stance_phase.

Firstly, the three-axis angular velocity data is selected to perform the quaternion operation to acquire the Euler angle euler_{z} in the Z-axis direction. A waveform of the Euler angle euler_{z} in the Z-axis direction is shown in 6a of FIG. 6, which reflects a pitch angle during a motion process.

Then, data of the Euler angle euler_{z} in the Z-axis direction is extracted when the state variable stationary is 0 to acquire several data segments eulerᵢ (i=1, 2, 3, . . . , step_n). A waveform of the state variable stationary is shown in 6b of FIG. 6, thereby acquiring the Euler angle euler_{z} in the Z-axis direction when the feet is swinging.

A heel landing angle angle_heel_strike is acquired through the following method.

A maximum value is extracted in each of the data segments eulerᵢ and an average value is calculated to acquire a heel landing angle angle_heel_strike in the gait parameters.

It is represented through a formula:

$$angle\_heel \_strike = \frac{1}{step\_n} \times {\sum }_{i = 1}^{step \_ n} max ( {euler}_{i} ) .$$

A toe off ground angle angle_toe_off is acquired through the following method.

A minimum value is extracted in each of the data segments eulerᵢ and an average value is calculated to acquire a toe off ground angle angle_toe_off in the gait parameters;

It is represented through a formula:

$$angle\_toe \_off = \frac{1}{step\_n} \times {\sum }_{i = 1}^{step \_ n} min ( {euler}_{i} ) .$$

A swinging phase swing_phase is acquired through the following method.

A difference value is calculated between an index idx_{angle_heel_strike} of a heel landing point and an index idx_{angle_toe_off} of a toe off point for each step to acquire a data length during a feet swinging process, and then a size of the data length during the feet swinging process as a percentage of an overall data length Nᵢ for each step is calculated to acquire a swinging phase swing_phase in the gait parameters, that is, a state of the feet stepping forward.

It is represented through a formula:

$$swing\_phase = \frac{1}{step\_n} \times {\sum }_{i = 1}^{step \_ n} \frac{{idx}_{i , angle \_ heel \_ strike} - {idx}_{i , angle \_ toe \_ off}}{{N}_{i}} .$$

A standing phase stance_phase is acquired through the following method.

A size of remaining data after removing the data length during the feet swinging process as a percentage of the overall data length Nᵢ for each step is calculated to acquire a standing phase stance_phase in the gait parameters, that is, a state of the feet stepping on ground and preparing to take a step.

It is represented through a formula:

$$stance\_phase = 1 - swing\_phase .$$

In one embodiment of the present disclosure, a method of acquiring the gait parameters related to the feet tremor includes:
performing a Fourier transform on the three-axis acceleration data and the three-axis angular velocity data to acquire 6 frequency domain data;
selecting a maximum frequency value of each frequency domain data within a set frequency threshold range to acquire 6 maximum frequency values;
counting the number of occurrences of each maximum frequency value, and selecting a maximum frequency value with the most occurrences as a tremor frequency; and
if multiple maximum frequency values occur the most frequently and are the same, taking their average value as the tremor frequency.

Based on a same inventive concept, the embodiments of the present disclosure further provide a gait analysis system.

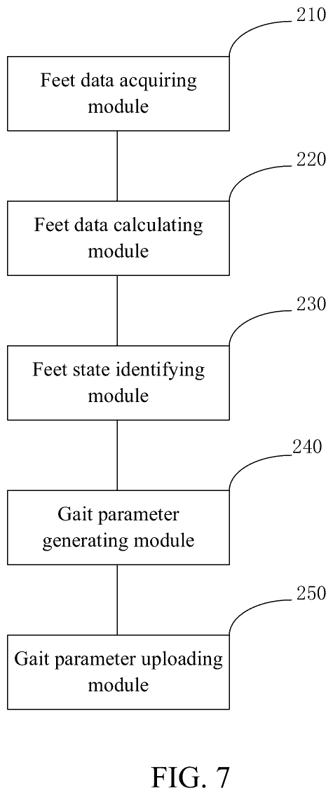

Referring to FIG. 7, the system includes:
a feet data acquiring module 210 configured to acquire three-axis acceleration data and three-axis angular velocity data collected by an inertial sensor;
a feet data calculating module 220 configured to perform a quaternion operation on the three-axis acceleration data and the three-axis angular velocity data to acquire three-axis acceleration and a three-axis Euler angle corresponding to a ground coordinate system;
a feet state identifying module 230 configured to identify a swinging state and a stationary state of feet;
a gait parameter generating module 240 configured to analyze based on the three-axis acceleration, the three-axis Euler angle, and the swinging state and the stationary state of the feet to acquire gait parameters related to the straight walking process, a turning processing, a feet to ground angle, and a feet tremor; and
a gait parameter uploading module 250 configured to upload types of the gait parameters and numerical values corresponding to the gait parameters to a server.

In one embodiment of the present disclosure the feet state identifying module 230 is specifically configured to:
preprocess the three-axis acceleration to acquire an acceleration signal; and
set a critical action threshold, identify a process that the acceleration signal exceeds the critical action threshold as the swinging state, identify a process that the acceleration signal is below the critical action threshold as the stationary state, and represent the critical action threshold using a state variable stationary, where when the state variable stationary is 1, it indicates that the feet are in the stationary state, and when the state variable stationary is 0, it indicates that the feet are in the swinging state.

In one embodiment of the present disclosure, the feet state identifying module 230 preprocesses the three-axis acceleration to acquire the acceleration signal includes:
calculating a vector sum of the three-axis acceleration to acquire an original acceleration signal;
performing a high-pass filtering process on the original acceleration signal with a cutoff frequency of 0.01 Hz to acquire an acceleration signal filtered once; and
performing a low-pass filtering process with a cutoff frequency of 5 Hz to acquire a final acceleration signal after performing an absolute value calculation on the acceleration signal filtered once.

In one embodiment of the present disclosure, a method of acquiring the gait parameters related to the straight walking process in the gait parameter generating module 240 includes:
performing a first-order differential operation on the state variable stationary to acquire a differential vector stationary_{diff}; identifying the differential vector stationary diff with a numerical value of −1 as a starting moment for each step, identifying the differential vector stationary diff with a numerical value of 1 as an ending moment for each step, and respectively counting the number of differential vectors stationary_{diff} with numerical values of −1 and 1; and selecting a minimum value from a counting result to acquire the number of steps step_n in the gait parameters;
selecting a starting point idxₛₜₐᵣₜ where all numerical values of the differential vector stationary_{diff} are −1 and an ending point idx_{end} where all numerical values of the differential vector stationary_{diff} are 1; calculating a velocity vel from the starting point idxₛₜₐᵣₜ to the ending point idx_{end}; calculating a starting position pos_{i,start} for each step based on the velocity vel at each moment before the starting point idxₛₜₐᵣₜ, and calculating an ending position pos_{i,end} for each step based on the velocity vel at each moment before the ending pointidx_{end}; and calculating a difference value between the ending position pos_{i,end} and the starting position pos_{i,start} to acquire a step lengthstep_length in the gait parameters;
calculating a data length from a starting point idx_{i,start} for each step to a starting point idx_{i+1,start} for a next step during a swinging process to acquire a data length Nᵢ for each step; calculating a ratio of the data length Nᵢ to a sampling frequency fs for each step and taking an average value to acquire a swinging period step_T; and calculating a reciprocal of the swinging period step_T to acquire a step frequency step_freq in the gait parameters;
calculating a ratio of a total step lengthstep_length to a total data length N at the sampling frequency fs within unit time to acquire a step velocity step_vel in the gait parameters; and
calculating a ratio of a standard deviation of all step lengths step_length to a step lengthstep_length to acquire a gait variation coefficient step_SD in the gait parameters.

In one embodiment of the present disclosure, a method of acquiring the gait parameters related to the turning process in the gait parameter generating module 240 includes:
selecting the three-axis angular velocity data to perform the quaternion operation to acquire an Euler angle eulerₓ in an X-axis;
performing a first-order differential operation on the Euler angle eulerₓ to acquire a differential Euler angle euler_{diff};
extracting all maximum points idx_peaksₚₒₛ and minimum points idx_peaks_{neg} in the differential Euler angle euler_{diff};
analyzing based on all the maximum points idx_peaksₚₒₛ and the minimum points idx_peaks_{neg} in the differential Euler angle euler_{diff} to acquire the starting point idxₛₜₐᵣₜ and the ending point idx_{end} for each step;
calculating a difference value between an Euler angle euler (idx_{end}) corresponding to the ending point idx_{end} and an Euler angle euler (idxₛₜₐᵣₜ) corresponding to the starting point idxₛₜₐᵣₜ to acquire an angle difference value Δ; removing the maximum points idx_peaksₚₒₛ and the minimum points idx_peaks_{neg} if an absolute value of the angle difference value Δ is lower than a first threshold; removing the minimum points idx_peaks_{neg} if an absolute value of the angle difference value Δ is higher than a second threshold; traversing the starting pointsidxₛₜₐᵣₜ and the ending point idx_{end} for each step acquired with a rule of removing the maximum points idx_peaksₚₒₛ, and counting a total number of remaining maximum points idx_peaksₚₒₛ and minimum points idx_peaks_{neg} to acquire the number of turning steps turn_n in the gait parameters if an absolute value of the angle difference value Δ is lower than a third threshold;
calculating a sum of the difference value between the Euler angle euler (idx_{end}) corresponding to the ending point idx_{end} and the Euler angle euler (idxₛₜₐᵣₜ) corresponding to the starting point idxₛₜₐᵣₜ for each of the number of turning steps turn_n and taking an average value to acquire a turning angle angle_turn in the gait parameters; and
calculating a ratio of the number of data points Nᵢ in a turning process for each of the number of turning steps turn_n to the sampling frequency fs to acquire turning time for each step; and calculating a ratio of the turning angle angle_turn to the turning time for each step and taking an average value to acquire a turning velocity angle_turn_vel in the gait parameters.

In one embodiment of the present disclosure, analyzing based on all the maximum points idx_peaksₚₒₛ and the minimum points idx_peaks_{neg} in the differential Euler angle euler_{diff} to acquire the starting point idxₛₜₐᵣₜ and the ending point idx_{end} for each step includes:
deleting an extreme point with a peak value less than a first peak threshold in the maximum points idx_peaksₚₒₛ and an extreme point with a peak value greater than a second peak threshold in the minimum points idx_peaks_{neg};
dividing the maximum points idx_peaksₚₒₛ or the minimum points idx_peaks_{neg} with a period interval less than a set period threshold into a group, and retaining the maximum points idx_peaksₚₒₛ or the minimum points idx_peaks_{neg} with a highest absolute peak value in each group;
selecting a data segment between any maximum point idx_peaksₚₒₛ and any minimum point idx_peaks_{neg} as a gait determination segment,
where if there are consecutive data points in the gait determination segment where a value of the differential Euler angle euler_{diff} is less than a fourth threshold and the number is less than N, then selected maximum points idx_peaksₚₒₛ and minimum points idx_peaks_{neg} are generated in a same step;
searching left for consecutive data points where the value of the differential Euler angle euler_{diff} is less than the fourth threshold and the number exceeds M based on the maximum points idx_peaksₚₒₛ or the minimum points idx_peaks_{neg} sorted first, and selecting a data point discovered first as the starting point idxₛₜₐᵣₜ for this step; and
searching right for consecutive data points where the value of the differential Euler angle euler_{diff} is less than the fourth threshold and the number exceeds M based on the maximum points idx_peaksₚₒₛ or the minimum points idx_peaks_{neg} sorted last, and selecting a data point discovered first as the ending point idx_{end} for this step.

In one embodiment of the present disclosure, a method of acquiring the gait parameters related to the feet to ground angle in the gait parameter generating module 240 includes:
selecting the three-axis angular velocity data to perform the quaternion operation to acquire the Euler angle euler_{z} in the Z-axis direction;
extracting data of the Euler angle euler_{z} in the Z-axis direction when the state variable stationary is 0 to acquire several data segments eulerᵢ (i=1, 2, 3, . . . , step_n);
extracting a maximum value in each of the data segments eulerᵢ and calculating an average value to acquire a heel landing angle angle_heel_strike in the gait parameters;
extracting a minimum value in each of the data segments eulerᵢ and
calculating an average value to acquire a toe off ground angle angle_toe_off in the gait parameters;
calculating a difference value between an index idx_{angle_heel_strike} of a heel landing point and an index idx_{angle_toe_off} of a toe off point for each step to acquire a data length during a feet swinging process, and then calculating a size of the data length during the feet swinging process as a percentage of an overall data length Nᵢ for each step to acquire a swinging phase swing_phase in the gait parameters; and
calculating a size of remaining data after removing the data length during the feet swinging process as a percentage of the overall data length Nᵢ for each step to acquire a standing phase stance_phase in the gait parameters.

In one embodiment of the present disclosure, a method of acquiring the gait parameters related to the feet tremor in the gait parameter generating module 240 includes:
performing a Fourier transform on the three-axis acceleration data and the three-axis angular velocity data to acquire 6 frequency domain data;
selecting a maximum frequency value of each frequency domain data within a set frequency threshold range to acquire 6 maximum frequency values;
counting the number of occurrences of each maximum frequency value, and selecting a maximum frequency value with the most occurrences as a tremor frequency; and
if multiple maximum frequency values occur the most frequently and are the same, taking their average value as the tremor frequency.

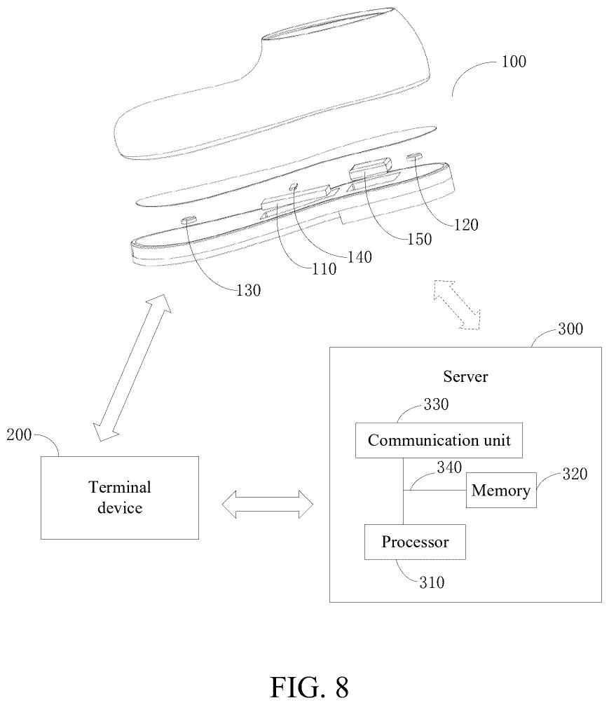

The embodiments of the present disclosure further provide an apparatus for improving a walking obstacle using the above gait analysis method. As shown in FIG. 8, the apparatus includes shoes 100, a terminal device 200, and a server 300.

The shoes 100 have an inertial sensor 110, a pressure sensor 120, a vibration element 130, an electrical stimulation element 140, and a communication module 150 built in.

The inertial sensor 110 is configured to collect three-axis acceleration data and three-axis angular velocity data, and the inertial sensor 110 generally integrates an accelerometer and a gyroscope.

The pressure sensor 120 is configured to collect pressure data.

The communication module 150 is configured to establish a communication link with the terminal device, and transmit the three-axis acceleration data, the three-axis angular velocity data, and the pressure data to the terminal device 200.

The server 300 is provided with a processor 310, a memory 320, and a communication unit 330, where the memory 320 is configured to store a program, and the three-axis acceleration data, the three-axis angular velocity data, and the pressure data from the terminal device 200, the processor 310 calls the program stored in the memory 320 to perform all steps in the gait analysis method in the above embodiments, and the communication unit 330 is configured to establish the communication link with the terminal device 200.

The terminal device 200 controls the vibration element 130 and the electrical stimulation element 140, where when a patient experiences an abnormal gait, the terminal device 200 controls the vibration element 130 to output vibration stimulation and/or the electrical stimulation element 140 to output electrical stimulation.

It should be noted that the processor 310, the memory 320, and the communication unit 330 communicate with each other through a bus 340.

Those ordinary skilled in the art should understand that the memory 320 includes but is not limited to a random access memory (referred to as RAM), a read only memory (referred to as ROM), a programmable read only memory (referred to as PROM), an erasable programmable read only memory (referred to as EPROM), an electric erasable programmable read only memory (referred to as EEPROM), and the like. Among them, the memory 320 is configured to store the program, and the processor 310 executes the program after receiving an execution instruction. Further, a software program and a module in the above memory 320 may also include an operating system, which may include various software components and/or drivers for managing system tasks such as memory management, storage device control, and power supply management, and may communicate with various hardware or software components to provide an operating environment for other software components.

The processor 310 may be a circuit chip with signal processing capabilities. The above processor 310 may be a general purpose processor, including a central processing unit (referred to as CPU), a network processor (referred to as NP), and the like. The disclosed methods, steps, and logical diagrams in the embodiments of the present disclosure may be implemented or performed. The general purpose processor may be a microprocessor or any conventional processor.

In practical applications, the pressure sensor 120 and the inertia sensor 110 inside the shoes 100 correspond to transmit the pressure data, the three-axis acceleration data, and the three-axis angular velocity data to the server 300 through the terminal device 200. The terminal device 200 may use a tablet, a mobile phone, a computer, a HUB, and the like, and the server 300 may use a local server or a cloud server. After the server 300 receives the data, a data analysis is performed to acquire gait parameters. Next, the pressure data and the gait parameters are used to identify whether the patient has the walking obstacle. If there is no walking obstacle, the shoes 100 are merely used to monitor and collect the data. If there is a walking obstacle, different stimulation modes are selected through the terminal device 200 to provide different stimulation methods for the shoes 100. Specifically, the terminal device 200 is embedded with a control program that sends a control instruction through wireless communication (such as Bluetooth) to control the vibration element 130 and the electrical stimulation element 140, so that the vibration element 130 and the electrical stimulation element 140 perform a corresponding action according to a corresponding control instruction to trigger a corresponding stimulation.

More specifically, the walking obstacle includes an abnormal gait, a tremor, and a freezing gait. When the patient experiences any of the above three walking obstacle symptoms, the vibration stimulation and/or the electrical stimulation is provided to the patient.

An identification method of the abnormal gait is as follows. The above method of calculating the step length is used, the number of steps and the step length are calculated based on the uploaded gait parameters, and an average value step_length_mean and a standard deviation step_length_sd of the last 10 steps are calculated (if a new step is generated, update the average value and the standard deviation). If a new step is generated in a latest determination segment, the step length of this step is calculated, and then whether the step length is greater than the average value plus three times the standard deviation, or less than the average value minus three times the standard deviation is determined. If it is, this step is marked, and if 5 consecutive steps are marked, itis determined that the abnormal gait has occurred, and if 5 consecutive steps are not marked, it is determined that no abnormal gait has occurred.

An identification method of the tremor is as follows. Based on the uploaded gait parameters, every 6 seconds are divided into a segment with a data length of 300, which serves as a basic determination segment. For each new 1 second of data collected, the newly collected data is reconstituted with the data of last 5 seconds of the previous segment to form a next determination segment, and so on. The tremor frequency in each determination segment is calculated using the above method. If the value is not 0, it is marked once. If 5 consecutive determination segments are marked, it is determined that the tremor has occurred, and if 5 consecutive determination segments are not marked, it is determined that no tremor has occurred.

An identification method of the frozen gait is as follows. The data length for determining a tremor is shortened to 2 seconds to acquire several determination segments, and if the frozen gait has occurred in these determination segments, they are marked. If 5 consecutive determination segments are marked, it is determined that the frozen gait has occurred, and if 5 consecutive determination segments are not marked, it is determined that no frozen gait has occurred.

The stimulation mode provides three types of stimulation modes, including no stimulation mode, adaptive stimulation mode, and continuous stimulation mode. These three stimulation modes may be autonomously selected according to an actual situation. The following will explain these three stimulation modes.

The no stimulation mode means no stimulation is generated for the patient to select independently.

The adaptive stimulation mode involves intermittent stimulation based on the actual situation. In this mode, when the gait of the patient is abnormal, stimulation has occurred when the shoes are stepped down, and no stimulation has occurred when the shoes are lifted up. Specifically, when a numerical value of the pressure data is greater than 600, it is determined that the shoes have been lifted up and no stimulation will be generated at this time, and when a numerical value of the pressure data is less than a threshold of 600, it is determined that the shoes have been stepped down, and the stimulation will be generated at this time. The stimulation method may be selected from either the vibration stimulation or the electrical stimulation, or both the vibration stimulation and the electrical stimulation may be selected at the same time.

The continuous stimulation mode refers to uninterrupted continuous stimulation. In this mode, when the gait of the patient is abnormal, the stimulation is performed regardless of whether the shoes have been lifted up or stepped down. The stimulation method may be either the vibration stimulation or the electrical stimulation, or both the vibration stimulation and the electrical stimulation may be performed at the same time.

In the adaptive stimulation mode and the continuous stimulation mode, when the patient experiences any of three abnormal gaits of the abnormal gait, the tremor, and the freezing gait, the vibration stimulation and/or the electrical stimulation are provided to the patient, and no stimulation has occurred after the symptoms disappear.

More specifically, there are two pressure sensors. Among them, one pressure sensor is provided at front half of a sole and corresponds to a forefoot position of a wearer, and the other pressure sensor is provided at rear half of the sole and corresponds to a heel position of the wearer. When two pressure sensors are inactive at the same time, it is identified as “lifting up gait information”, thereby accurately activating the vibration stimulation. At the same time, in a process of generating “stepping down gait information”, overall time of transition from a heel to a forefoot during motion is included in the “stepping down gait information” under an action of two pressure sensors.

In another method, there are multiple pressure sensors, one part of the pressure sensors are provided vertically in a forefoot area and tilted upwards along a direction of a little toe towards a big toe, while the other part of the pressure sensors are distributed in a triangular and equidistant manner in a heel area. An aim is to adapt feet bones to be able to accurately acquire the pressure data from the patient. The other part of the pressure sensors are distributed in the triangular and equidistant manner in the heel area of the sole. By providing the pressure sensors in the forefoot area and the heel area of the sole, it is possible to accurately sense a “lifting up” state and a “stepping down” state of the patient while walking.

More specifically, the pressure sensors are provided in two rows in the forefoot area of the sole, with three pressure sensors in an upper row. The three pressure sensors are respectively a first pressure sensor, a second pressure sensor, and a third pressure sensor. The second pressure sensor is located between the first pressure sensor and the third pressure sensor. There are two pressure sensors in a lower row. The two pressure sensor are respectively a fourth pressure sensor and a fifth pressure sensor, and positions of the fourth sensor and fifth pressure sensor provided in the lower row are respectively opposite to positions of the first pressure sensor and the third pressure sensor provided in the upper row. Through the above settings, points for acquiring the pressure data of the patient can be expanded, allowing the doctor to fully grasp gait information of the patient and provide a more accurate treatment plan.

More specifically, in order to form a three-dimensional vibration stimulation with directivity, the vibration element in the shoes of the present disclosure uses a vibration motor. There are multiple vibration motors, and directivity of vibration waves of the multiple vibration motors is corresponding to an ankle position of the wearer, and the multiple vibration motors form regional resonant vibration with the directivity by adjusting a vibration frequency of each of the vibration motors. More specifically, the directivity of the vibration waves of the multiple vibration motors may correspond to an inner ankle position of the wearer, or correspond to an outer ankle position of the wearer, or directly point to a proprioceptor receptorposition of the feet of the wearer.

In practical applications, specifically, there are 4 vibration motors, including a first vibration motor, a second vibration motor, a third vibration motor, and a fourth vibration motor, where the first vibration motor is provided at a position corresponding to an arch, the second vibration motor is provided at a position corresponding to a calcaneus, the third vibration motor is provided at a position corresponding to a talus, and the fourth vibration motor is provided at a position corresponding to a lower end of a tibia, and vibration waves of the first vibration motor, the second vibration motor, the third vibration motor, and the fourth vibration motor point to the lower end of the tibia at the same time. Through the above settings, the vibration generated by each vibration motor mainly acts on a cartilage contact area at an ankle, that is, a tendon, a ligament and a cartilage in a combined area of the tibia, the calcaneus and the talus, and the above position is a main attachment organ of the proprioceptive receptor. The vibration motors located at different positions are made to produce a same vibration effect on a same position of the patient. The vibration motors are used to compensate for the stimulation of motor senses of the patient through a mechanical vibration method to reconstruct a complete motion control loop. In addition, rated speed of each of the vibration motors is controlled within a range of 1-15000 RPM.

The electrical stimulation element may be built into the shoes according to the above setting method of the vibration motor, and will not be further explained herein.

The shoes in the above embodiments further have a remote control receiving module built in, the remote control receiving module is connected to a processor, the remote control receiving module is configured to establish a communication link between the processor and a remote control transmitting module, and the wearer sends a remote control instruction to the vibration element and the electrical stimulation element through the remote control transmitting module. The remote control receiving module is an infrared receiver built into the shoes, and the remote control transmitting module is an infrared remote control (the infrared remote control is used in combination with the remote control receiving module inside the shoes). An operation panel of the infrared remote control includes a start function key, a stop function key, a stimulation enhancement function key, and a stimulation attenuation function key for controlling the processor. In this way, the patient may also adjust the frequency of the vibration stimulation through the remote control even when the vibration stimulation or the electrical stimulation is not significant enough.

In order to increase a transmission distance of the remote control, operational convenience is improved, and adaptability is enhanced. A function keyboard can be integrated on a human-computer interactive interface of the terminal device through the remote control, and the function keyboard sends a control signal in a touch manner to remotely control activation and deactivation of the vibration motor and/or the electrical stimulation element, and the function keyboard is provided with a number key, a reserving key, a frequency adjusting key, an overall group controlling key, a confirming key, a closing key, an automatic and manual switching key, and a local group controlling key to implement various control modes. The patient can receive treatment at home through this method.

The shoes in the above embodiments further have an energy storing module built in, which is respectively connected to a processor, a gait sensing module, and a vibration stimulating module. The energy storing modules provide energy source for the processor, the gait sensing module, and the vibration stimulating module. The energy storing module includes a lithium battery and a charging interface, with the charging interface using a min USB interface or a USB magnetic interface. Usage time is about 8 hours, and charging time is 2 hours, which meets travel demands.

The present disclosure further provides a computer readable storage medium, including a program, where the program, when performed by a processor, is configured to perform a gait analysis method according to any of the above method embodiments.

Those skilled in the art should understand that all or part of steps for implementing the above method embodiments may be completed through hardware related to program instructions. The above program may be stored in a computer readable storage medium. When the program is executed, steps of the above method embodiments also executed. Moreover, the above storage medium includes various media such as a ROM, a RAM, a magnetic disk, or an optical disk that may store a program code, and a specific type of the medium is not limited in the present disclosure.

The above are merely preferred specific implementation manners in the present disclosure, but a protection scope of the present disclosure is not limited thereto. Any variations or replacements that may be easily conceived of by those skilled that are familiar with the art within a technical scope disclosed in the present disclosure also fall within the protection scope of the present disclosure. Therefore, the protection scope of the present disclosure should be based on the protection scope of the claims.

The above are merely the embodiments of the present disclosure and are not intended to limit the present disclosure. For those skilled in the art, the present disclosure may have various modifications and changes. Any modifications, equivalent replacements, improvements, and the like made within spirits and principles of the present disclosure should fall within a scope of the claims of the present disclosure.

### INDUSTRIAL APPLICABILITY

A gait analysis method and an apparatus for improving a walking obstacle using the method provided by the present disclosure are suitable for a patient with the walking obstacle, which can provide objective and comprehensive motion data of a patient's feet, thereby providing an objective basis for formulating a rehabilitation treatment plan and evaluating rehabilitation efficacy. Collection of the motion data is not limited by a venue, which may reduce a burden on a doctor and a patient. The formed product may be mass-produced and used in industry.

1. A gait analysis method, comprising the following steps:
acquiring three-axis acceleration data and three-axis angular velocity data collected by an inertial sensor;
performing a quaternion operation on the three-axis acceleration data and the three-axis angular velocity data to acquire three-axis acceleration and a three-axis Euler angle corresponding to a ground coordinate system;
identifying a swinging state and a stationary state of feet;
analyzing based on the swinging state and the stationary state to acquire gait parameters related to a straight walking process;
analyzing based on the three-axis acceleration, the three-axis Euler angle, and the swinging state and the stationary state of the feet to acquire gait parameters related to the straight walking process, a turning processing, a feet to ground angle, and a feet tremor; and
uploading types of the gait parameters and numerical values corresponding to the gait parameters to a server.

2. The gait analysis method according to claim 1, wherein identifying the swinging state and the stationary state of the feet comprises:
setting a critical action threshold and representing it using a state variable stationary, wherein when the state variable stationary is 1, it indicates that the feet are in the stationary state, and when the state variable stationary is 0, it indicates that the feet are in the swinging state;
preprocessing the three-axis acceleration to acquire an acceleration signal, wherein if the acceleration signal exceeds the critical action threshold, it is considered to be in the swinging state, and if the acceleration signal is below the critical action threshold, it is considered to be in the stationary state, and a first state variable stationary1 is acquired;
acquiring pressure data collected by a pressure sensor, wherein if the pressure data exceeds the critical action threshold, it is considered to be in the swinging state, and if the pressure data below the critical action threshold, it is considered to be in the stationary state, and a second state variable stationary2 is acquired; and
performing an OR operation on the first state variable stationary1 and the second state variable stationary2, and identifying the swinging state and the stationary state of the feet based on an operation result.

3. The gait analysis method according to claim 2, wherein preprocessing the three-axis acceleration to acquire the acceleration signal comprises:
calculating a vector sum of the three-axis acceleration to acquire an original acceleration signal;
performing a high-pass filtering process on the original acceleration signal with a cutoff frequency of 0.01 Hz to acquire an acceleration signal filtered once; and
performing a low-pass filtering process with a cutoff frequency of 5 Hz to acquire a final acceleration signal after performing an absolute value calculation on the acceleration signal filtered once.

4. The gait analysis method according to claim 2, wherein a method of acquiring the gait parameters related to the straight walking process comprises:
performing a first-order differential operation on the state variable stationary to acquire a differential vector stationary_{diff}; identifying the differential vector stationary_{diff} with a numerical value of −1 as a starting moment for each step, identifying the differential vector stationary_{diff} with a numerical value of 1 as an ending moment for each step, and respectively counting the number of differential vectors stationary_{diff} with numerical values of −1 and 1; and selecting a minimum value from a counting result to acquire the number of steps step_n in the gait parameters;
selecting a starting point idxₛₜₐᵣₜ where all numerical values of the differential vector stationary_{diff} are −1 and an ending point idx_{end} where all numerical values of the differential vector stationary_{diff} are 1; calculating a velocity velfrom the starting point idxₛₜₐᵣₜ to the ending point idx_{end}; calculating a starting position pos_{i,start} for each step based on the velocity vel at each moment before the starting point idxₛₜₐᵣₜ, and calculating an ending position pos_{i,end} for each step based on the velocity vel at each moment before the ending pointidx_{end}; and calculating a difference value between the ending position pos_{i,end} and the starting position pos_{i,start} to acquire a step lengthstep_length in the gait parameters;
calculating a data length from a starting point idx_{i,start} for each step to a starting point idx_{i+1,start} for a next step during a swinging process to acquire a data length Nᵢ for each step; calculating a ratio of the data length Nᵢ to a sampling frequency fs for each step and taking an average value to acquire a swinging period step_T; and calculating a reciprocal of the swinging period step_T to acquire a step frequency step_freq in the gait parameters;
calculating a ratio of a total step lengthstep_length to a total data length N at the sampling frequency fs within unit time to acquire a step velocity step_vel in the gait parameters; and
calculating a ratio of a standard deviation of all step lengths step_length to a step lengthstep_length to acquire a gait variation coefficient step_SD in the gait parameters.

5. The gait analysis method according to claim 1, wherein a method of acquiring the gait parameters related to the turning process comprises:
selecting the three-axis angular velocity data to perform the quaternion operation to acquire an Euler angle eulerₓ in an X-axis;
performing a first-order differential operation on the Euler angle eulerₓ to acquire a differential Euler angle euler_{diff};
extracting all maximum points idx_peaksₚₒₛ and minimum points idx_peaks_{neg} in the differential Euler angle euler_{diff};
analyzing based on all the maximum points idx_peaksₚₒₛ and the minimum points idx_peaks_{neg} in the differential Euler angle euler_{diff} to acquire the starting point idxₛₜₐᵣₜ and the ending point idx_{end} for each step;
calculating a difference value between an Euler angle eulerₓ (idx_{end}) corresponding to the ending point idx_{end} and an Euler angle eulerₓ (idxₛₜₐᵣₜ) corresponding to the starting point idxₛₜₐᵣₜ to acquire an angle difference value Δ; removing the maximum points idx_peaksₚₒₛ and the minimum points idx_peaks_{neg} if an absolute value of the angle difference value Δ is lower than a first threshold; removing the minimum points idx_peaks_{neg} if an absolute value of the angle difference value Δ is higher than a second threshold; traversing the starting pointsidxₛₜₐᵣₜ and the ending point idx_{end} for each step acquired with a rule of removing the maximum points idx_peaksₚₒₛ, and counting a total number of remaining maximum points idx_peaksₚₒₛ and minimum points idx_peaks_{neg} to acquire the number of turning steps turn_n in the gait parameters if an absolute value of the angle difference value Δ is lower than a third threshold;
calculating a sum of the difference value between the Euler angle eulerₓ (idx_{end}) corresponding to the ending point idx_{end} and the Euler angle eulerₓ (idxₛₜₐᵣₜ) corresponding to the starting point idxₛₜₐᵣₜ for each of the number of turning steps turn_n and taking an average value to acquire a turning angle angle_turn in the gait parameters; and
calculating a ratio of the number of data points Nᵢ in a turning process for each of the number of turning steps turn_n to the sampling frequency fs to acquire turning time for each step; and calculating a ratio of the turning angle angle_turn to the turning time for each step and taking an average value to acquire a turning velocity angle_turn_vel in the gait parameters.

6. The gait analysis method according to claim 4, wherein analyzing based on all the maximum points idx_peaksₚₒₛ and the minimum points idx_peaks_{neg} in the differential Euler angle euler_{diff} to acquire the starting point idxₛₜₐᵣₜ and the ending point idx_{end} for each step comprises:
deleting an extreme point with a peak value less than a first peak threshold in the maximum points idx_peaksₚₒₛ and an extreme point with a peak value greater than a second peak threshold in the minimum points idx_peaks_{neg};
dividing the maximum points idx_peaksₚₒₛ or the minimum points idx_peaks_{neg} with a period interval less than a set period threshold into a group, and retaining the maximum points idx_peaksₚₒₛ or the minimum points idx_peaks_{neg} with a highest absolute peak value in each group;
selecting a data segment between any maximum point idx_peaksₚₒₛ and any minimum point idx_peaks_{neg} as a gait determination segment,
wherein if there are consecutive data points in the gait determination segment where a value of the differential Euler angle euler_{diff} is less than a fourth threshold and the number is less than N, then selected maximum points idx_peaksₚₒₛ and minimum points idx_peaks_{neg} are generated in a same step;
determining an order of the maximum points idx_peaksₚₒₛ and the minimum points idx_peaks_{neg} in the same step;
searching left for consecutive data points where the value of the differential Euler angle euler_{diff} is less than the fourth threshold and the number exceeds M based on the maximum points idx_peaksₚₒₛ or the minimum points idx_peaks_{neg} sorted first, and selecting a data point discovered first as the starting point idxₛₜₐᵣₜ for this step; and
searching right for consecutive data points where the value of the differential Euler angle euler_{diff} is less than the fourth threshold and the number exceeds M based on the maximum points idx_peaksₚₒₛ or the minimum points idx_peaks_{neg} sorted last, and selecting a data point discovered first as the ending point idx end for this step.

7. The gait analysis method according to claim 2, wherein a method of acquiring the gait parameters related to the feet to ground angle comprises:
selecting the three-axis angular velocity data to perform the quaternion operation to acquire the Euler angle euler_{z} in the Z-axis direction;
extracting data of the Euler angle euler_{z} in the Z-axis direction when the state variable stationary is 0 to acquire several data segments eulerᵢ (i=1, 2, 3, . . . , step_n);
extracting a maximum value in each of the data segments eulerᵢ and calculating an average value to acquire a heel landing angle angle_heel_strike in the gait parameters;
extracting a minimum value in each of the data segments eulerᵢ and calculating an average value to acquire a toe off ground angle angle_toe_off in the gait parameters;
calculating a difference value between an index idx_{angle_heel_strike} of a heel landing point and an index idx_{angle_toe_off} of a toe off point for each step to acquire a data length during a feet swinging process, and then calculating a size of the data length during the feet swinging process as a percentage of an overall data length Nᵢ for each step to acquire a swinging phase swing_phase in the gait parameters; and
calculating a size of remaining data after removing the data length during the feet swinging process as a percentage of the overall data length Nᵢ for each step to acquire a standing phase stance_phase in the gait parameters.

8. The gait analysis method according to claim 2, wherein a method of acquiring the gait parameters related to the feet tremor comprises:
performing a Fourier transform on the three-axis acceleration data and the three-axis angular velocity data to acquire 6 frequency domain data;
selecting a maximum frequency value of each frequency domain data within a set frequency threshold range to acquire 6 maximum frequency values;
counting the number of occurrences of each maximum frequency value, and selecting a maximum frequency value with the most occurrences as a tremor frequency; and
if multiple maximum frequency values occur the most frequently and are the same, taking their average value as the tremor frequency.

9. A gait analysis system, comprising:
a feet data acquiring module configured to acquire three-axis acceleration data and three-axis angular velocity data collected by an inertial sensor;
a feet data calculating module configured to perform a quaternion operation on the three-axis acceleration data and the three-axis angular velocity data to acquire three-axis acceleration and a three-axis Euler angle corresponding to a ground coordinate system;
a feet state identifying module configured to identify a swinging state and a stationary state of feet;
a gait parameter generating module configured to analyze based on the three-axis acceleration, the three-axis Euler angle, and the swinging state and the stationary state of the feet to acquire gait parameters related to the straight walking process, a turning processing, a feet to ground angle, and a feet tremor; and
a gait parameter uploading module configured to upload types of the gait parameters and numerical values corresponding to the gait parameters to a server.

10. (canceled)

11. An apparatus for improving a walking obstacle,
comprising shoes, a terminal device, and a server, wherein
the shoes have an inertial sensor, a pressure sensor, a vibration element, an electrical stimulation element, and a communication module built in, wherein
the inertial sensor is configured to collect three-axis acceleration data and three-axis angular velocity data;
the pressure sensor is configured to collect pressure data;
the communication module is configured to establish a communication link with the terminal device, and transmit the three-axis acceleration data, the three-axis angular velocity data, and the pressure data to the terminal device;
the server is provided with a processor, a memory, and a communication unit, wherein the memory is configured to store a program, and the three-axis acceleration data, the three-axis angular velocity data, and the pressure data from the terminal device, the processor calls the program stored in the memory to perform the gait analysis method according to claim 1, and the communication unit is configured to establish the communication link with the terminal device; and
the terminal device controls the vibration element and the electrical stimulation element, wherein when a patient experiences an abnormal gait, the terminal device controls the vibration element to output vibration stimulation and/or the electrical stimulation element to output electrical stimulation.

12. The apparatus for improving the walking obstacle according to claim 11, wherein there are two pressure sensors, one pressure sensor is provided at front half of a sole and corresponds to a forefoot position of a wearer, and the other pressure sensor is provided at rear half of the sole and corresponds to a heel position of the wearer.

13. The apparatus for improving the walking obstacle according to claim 11, wherein there are multiple pressure sensors, one part of the pressure sensors are provided vertically in a forefoot area and tilted upwards along a direction of a little toe towards a big toe, while the other part of the pressure sensors are distributed in a triangular and equidistant manner in a heel area.

14. The apparatus for improving the walking obstacle according to claim 11, wherein the vibration element uses a vibration motor, there are multiple vibration motors, and directivity of vibration waves of the multiple vibration motors is corresponding to an ankle position of the wearer, and the multiple vibration motors form regional resonant vibration with the directivity by adjusting a vibration frequency of each of the vibration motors.

15. The apparatus for improving the walking obstacle according to claim 14, wherein the directivity of the vibration waves of the multiple vibration motors corresponds to an inner ankle position of the wearer, or corresponds to an outer ankle position of the wearer, or directly points to a proprioceptor receptor position of the feet of the wearer.

16. The apparatus for improving the walking obstacle according to claim 15, wherein rated speed of each of the vibration motors is controlled within a range of 1-15000 RPM.

17. The apparatus for improving the walking obstacle according to claim 11, wherein there are 4 vibration motors, comprising a first vibration motor, a second vibration motor, a third vibration motor, and a fourth vibration motor, wherein the first vibration motor is provided at a position corresponding to an arch, the second vibration motor is provided at a position corresponding to a calcaneus, the third vibration motor is provided at a position corresponding to a talus, and the fourth vibration motor is provided at a position corresponding to a lower end of a tibia, and vibration waves of the first vibration motor, the second vibration motor, the third vibration motor, and the fourth vibration motor point to the lower end of the tibia at the same time.

18. The apparatus for improving the walking obstacle according to claim 11, wherein the shoes further have a remote control receiving module built in, the remote control receiving module is connected to a processor, the remote control receiving module is configured to establish a communication link between the processor and a remote control transmitting module, and the wearer sends a remote control instruction to the vibration element and the electrical stimulation element through the remote control transmitting module.

19. The apparatus for improving the walking obstacle according to claim 18, wherein the remote control transmitting module is an infrared remote control, and an operation panel of the infrared remote control comprises a start function key, a stop function key, a stimulation enhancement function key, and a stimulation attenuation function key for controlling the processor.

20. The apparatus for improving the walking obstacle according to claim 11, wherein a function keyboard is integrated on a human-computer interactive interface of the terminal device, and the function keyboard sends a control signal in a touch manner to remotely control activation and deactivation of the vibration motor and/or the electrical stimulation element, and the function keyboard is provided with a number key, a reserving key, a frequency adjusting key, an overall group controlling key, a confirming key, a closing key, an automatic and manual switching key, and a local group controlling key.
